SEQUENCE LISTING (5 sequences)

SEQ ID NO: 1 - DNA, 54 bp, Artificial Sequence
  Synthetic Construct
gggacgcgtcggcctggcacgtcggccgctgcggccaggcacccgatggcgttt

SEQ ID NO: 2 - DNA, 50 bp, Artificial Sequence
  Synthetic Construct
gccatcgggtgcctggccgcagcggccgacgtgccaggccgacgcgtccc

SEQ ID NO: 3 - DNA, 18 bp, Artificial Sequence
  Synthetic construct
gagctccaccgcggtggc

SEQ ID NO: 4 - DNA, 18 bp, Artificial Sequence
  Synthetic Construct
ggtaccgggccccccctc

SEQ ID NO: 5 - DNA, 226 bp, Artificial Sequence
  Synthetic construct
ggaaacagctatgaccatgattacgccaagctcgaaattaaccctcactaaagggaacaaaagctggagctccaccgcggtggcggccgctctagaactagtggatcccccgggctgcaggaattcgatatcaagcttatcgataccgtcgacctcgagggggggcccggtacccaattcgccctatagtgagtcgtattacaattcactggccgtcgttttacaa

(19) United States
(12) Patent Application Publication
(10) Pub. No.: US 2006/0094052 A1
(43) Pub. Date: May 4, 2006

(54) PCR-based substrate preparation for helicase assays

(76) Inventors: Zvi Kelman, Gaithersburg, MD (US); Jae-Ho Shin, Rockville, MD (US)

Correspondence Address: INTELLECTUAL PROPERTY / TECHNOLOGY LAW, PO BOX 14329, RESEARCH TRIANGLE PARK, NC, 27709, US

(21) Appl. No.: 11/265,954

(22) Filed: Nov. 3, 2005

Related U.S. Application Data
(60) Provisional application No. 60/624,571, filed on Nov. 3, 2004.

(51) Int. Cl. C12Q 1/68; C12P 19/34

(52) U.S. Cl. 435/6; 435/91.2

(57) ABSTRACT

The present invention relates to a PCR method for generating a labeled double stranded nucleotide sequence that upon digestion with a restriction nuclease generates a double stranded nucleotide sequence having either a 3′ or 5′ overhang of a ssDNA sequence that can serve as helicase substrate.

### CROSS REFERENCE TO RELATED APPLICATIONS

This application claims benefit of priority of U.S. provisional application No. 60/624,571 entitled “PCR-BASED SUBSTRATE PREPARATION FOR HELICASE ASSAY” filed on Nov. 3, 2004, the contents of which is incorporated by reference herein.

### GOVERNMENT RIGHTS

Not Applicable

### BACKGROUND OF THE INVENTION

1. Technical Field

The present invention relate generally to assays, and more particularly, to a PCR-based method for generating substrates for helicase assays.

2. Description of Related Art

Many biological processes, including DNA replication, recombination, repair and transcription, require the transient unwinding of duplex DNA. This task is handled by a group of enzymes, DNA helicases, which catalyze the unwinding of duplex DNA using energy derived from nucleoside triphosphate hydrolysis to melt the duplex. Helicases translocate along one strand of DNA and displace the complimentary strand (1,2) and have a specific polarity depending upon the single-stranded DNA (ssDNA) strand on which they move, either in the 3′→5′ or in the 5′→3′ direction.

To date, the most commonly used substrate for helicase assays use a short 32P-labeled oligonucleotide annealed to a longer ssDNA molecule, either single-stranded plasmid DNA (M13 or φX174) (3,4) or to a long oligonucleotide (5). The helicase unwinds the duplex region presented in these partial duplex substrates, yielding two ssDNA molecule with different sizes that can be resolved from the starting duplex substrate by electrophoresis followed by autoradiography [summarized in (6.7)].

This approach has several disadvantages for routine work, especially for laboratories that do not routinely perform such assays. When oligonucleotides are used, they have to be labeled, annealed to each other and then purified from an acrylamide gel to remove unincorporated nucleosides and any excess unannealed oligonucleotides. In addition, the length of the substrate is limited by the length of oligonucleotide that can be synthesized. When single-stranded plasmid DNA is used, a column (G-25 or G-50) is used to separate the labeled oligonucleotides from the substrate. To make a long substrate, an additional elongation step using DNA polymerase is needed.

The currently available techniques to generate substrates for helicase assays are fairly complicated and need some expertise not available in all laboratories. Thus, it would be advantageous to develop techniques that circumvent some of the problems encountered by the other conventional way to make helicase substrates.

### SUMMARY OF THE INVENTION

The present invention relates to a PCR method that generates a labeled product that upon digestion by a restriction enzyme can serve as helicase substrate, thereby making helicase substrate preparation simpler in comparison with other techniques and ease preparation of long DNA substrates.

In one aspect, the present invention relates to a helicase substrate comprising:
a nucleotide template/primer double stranded complex comprising a recognizable nucleotide sequence by a restriction nuclease that upon restriction nuclease digestion of the nucleotide template/primer complex will provide double stranded nucleotide sequence fragments with a 3′ end or 5′ end overhanging ssDNA strand.

In another aspect the present invention relates to a method for generating a helicase substrate for use in a helicase assay, the method comprising:
(a) providing a PCR reaction composition comprising a template nucleotide sequence and at least one primer;
(b) hybridizing the primer to the template nucleotide sequence to form a template/primer complex;
(c) separating the template/primer complex from unannealed template and primers;
(d) digesting the template/primer complex with a restriction enzyme; and
(e) separating double stranded fragments with a 3′ end or 5′ end overhang and using same as the helicase substrate in the helicase assay.

In another aspect, the present invention relates to a method for generating a helicase substrate for use in a helicase activity assay, the method comprising:
(a) providing a template nucleotide sequence having a recognition sequence for a restriction nuclease, wherein the restriction nuclease cuts in an offset fashion to produce ends having an overhanging piece of a single-stranded nucleotide sequence;
(b) providing a first and second primer having a nucleotide sequence that will anneal to the 5′ and 3′ of the template nucleotide sequence, wherein the primers are sufficiently complementary to the template nucleotide sequence to hybridize therewith such that a first extension sequence synthesized from the first primer, when separated from its complement, can serve as a template for synthesis of a second extension sequence of the second primer;
(c) amplifying the template nucleotide sequence by PCR amplification by combining at least the first and second primers, nucleotide bases and amplifying reagents to couple the nucleotide bases to the primers and generating the extension sequence complementary to the template nucleotide sequence to form a double stranded DNA sequence;
(d) contacting the double stranded DNA sequence with the restriction nuclease to form double stranded DNA sequences having a 5′ or 3′ overhanging single stranded DNA end; and
(e) using the double stranded DNA sequences having the 5′ or 3′ overhanging single stranded DNA end as a helicase substrate in a helicase assay.

The double stranded DNA sequences having either a 5′ end or 3′ overhanging end, preferably has an overhang from about 1 to 10 bases, and more preferably from about 3 to 5 bases.

Preferably, at least one of the primers is labeled with signal tag so that the double-stranded DNA sequence when used in a helicase assay allows for the detection of released helicase reaction product. Alternatively, labeled nucleotide bases may be used in lieu of the labeled primers, wherein the labeled nucleotide bases are incorporated into the extension sequences. Preferably, the primers or nucleotide bases are radiolabeled. Fluorescence and other conventional detection methods may also be used, so long as they are sufficiently sensitive and accurate for detection.

In yet another aspect, the present invention relates to a method for generating a helicase substrate for use in a helicase activity assay, the method comprising:
(a) treating a template nucleotide sequence with one oligonucleotide primer for each strand of the template nucleotide sequence, under hybridizing conditions such that an extension product of each primer is synthesized which is complementary to each strand to form an amplification solution, wherein said primer or primers are selected so as to be substantially complementary to each strand such that an extension sequence synthesized from one primer, when it is separated from its complement, can serve as a template for synthesis of an extension sequence of the other primer, and wherein the template nucleotide sequence has at least one recognition sequence for a restriction nuclease;
(b) treating the amplification solution under denaturing conditions to separate the primer extension sequences from their templates;
(c) treating the amplification solution with oligonucleotides primers such that a primer extension product is synthesized using each of the single strands produced in step (b) as a template, resulting in amplification of the template nucleic acid sequence; and
(d) adding to the product of step (c) a restriction nuclease to form double stranded nucleotide sequence fragments, wherein the restriction nuclease cuts in an offset fashion to produce ends having an overhanging piece of a single-stranded nucleotide sequence; and
(e) separating the double stranded nucleotide sequence fragments for use as helicase substrates in a helicase assay.

Importantly, the steps (b)-(c) may be repeated until the desired level of sequence amplification is obtained.

In another aspect, the present invention relates to helicase assay kit for the detection of activity of suspected helicases, the kit comprising:
(a) a template nucleotide sequence, a first and second primer, wherein the primers are substantially complementary to each strand of each specific nucleic acid sequence such that an extension sequence synthesized from one primer, when it is separated from its complement, can serve as a template for the synthesis of the extension product of the other primer;
(b) an agent for polymerization;
(c) four different nucleoside triphosphates; and
(d) a restriction nuclease, where the wherein the restriction nuclease cuts in an offset fashion to produce ends having an overhanging piece of a single-stranded nucleotide sequence, thereby providing a helicase substrate having either a 5′ or 3′ overhanging end and usable as a helicase substrate in the helicase assay to determine helicase activity of a suspected helicase whether it moves in either in the 3′→5′ or in the 5′→3′ direction.

Other features and advantages of the invention will be apparent from the following detailed description, drawings and claims.

### BRIEF DESCRIPTION OF THE FIGURES

FIG. 1 shows that M. thermautotrophicus MCM can initiate duplex DNA unwinding from a short 3′ single-stranded overhang. Two oligonucleotides were used to generate a helicase substrate with a 4-base ssDNA overhang. Helicase assays were performed as described in Material and Methods in 15 μl reactions with the indicated concentrations of protein and 10 fmol substrate. The ³²P-labeled oligonucleotide is marked with an asterisk. Lane 1, substrate only; lane 2, boiled substrate; lanes 3-5 contain 0.13, 0.40 and 1.2 pmol of proteins (as monomer), respectively. The percent displacement of the labeled oligonucleotide is indicated as %.

FIG. 2 is a schematic diagram for the procedures developed to generate a PCR-based substrate for helicase assays. Bold arrows are the PCR primers and ³²P is depicted by asterisks.

FIG. 3 shows substrates for helicase assays generated by PCR. A PCR product and its restriction fragment derivatives are shown. PCR reactions were performed as described in Materials and Methods with ³²P-labeled primer, and products were purified using the QIAquick PCR purification kit (Qiagen) (lane 1) and digest with PstI (lane 2), EcoRI (lane 3) and SmaI (lane 4) restriction enzymes.

FIG. 4 shows that PCR-generated substrates can be used for helicase assays.
(A) Helicase assays with the M. thermautotrophicus MCM helicase. Helicase assays were performed as described in Material and Methods with 10 fmol of substrate as indicated in the figure. Lanes 1, 6 and 11, substrate only; lanes 2, 7 and 12, boiled substrate; lanes 3-5, 8-10 and 13-15,containing 0.13, 0.40 and 1.2 pmol of proteins (as monomer), respectively. The ³²P-labeled strands are marked with an asterisk. The percent displacement of the labeled oligonucleotide is indicated as %.
(B) PCR-generated substrates can be used for helicases with different polarities. The helicase activity of several bacterial and viral helicases was determined using the PCR-based substrate containing either a 5′ or a 3′ single-stranded overhang. Helicase assays were performed as described in Material and Methods with 10 fmol of each substrate and 1.2 pmol of enzyme. ³²P-labeled strands are marked with an asterisk. Helicases with known 5′→3′ polarity are shown. Lane 1, substrate only; lane 2, boiled substrate; lane 3, SV-40 large T-antigen; lane 4, E. coli PriA; lane 5, E. coli Rep helicase; lane 6, E. coli RecG; lane 7, E. coli UvrD.
(C) Helicases with known 5′→3′ polarity are shown. Lane 1, substrate only; lane 2, boiled substrate; lane 3, E. coli RecQ; lane 4, E. coli RecG. The percent displacement of the labeled oligonucleotide is indicated as %.

### DETAILED DESCRIPTION OF THE INVENTION

The present invention provides a new way to generate helicase substrates using a PCR-based approach which circumvents some of the problems encountered by the other conventional ways to make helicase substrates. In the past several years, a large number of new helicases have been identified in laboratories working on different aspects of nucleic acid enzymology. The technique described herein may enable laboratories that do not routinely make helicase substrates to use readily available laboratory equipment and techniques to analyze putative helicases. It may also be used to generate helicase substrates containing specific DNA sequences, e.g. protein-binding sites, DNA sequences capable of forming Z-DNA, etc.

PCR Technology

Nucleic acid amplification generally proceeds via a particular protocol. One useful protocol is that set forth in U.S. Pat. No. 4,683,195, the contents of which are incorporated by reference herein for all purposes.

In general, the polymerase chain reaction involves the use of a pair of specific oligonucleotide primers to initiate DNA synthesis on a template nucleotide sequence. Two oligonucleotide primers are used for each double-stranded sequence to be amplified. The template nucleotide sequence is denatured into its complimentary strands. Each of the primers, which are sufficiently complementary to a portion of each strand of the template nucleotide sequence to hybridize with it, anneals to one of the strands. The primers are extended, using nucleosides in the sample and a polymerization agent, such as heat-stable Taq DNA polymerase. This results in the formation of complementary primer extension sequences, which are hybridized to the complementary strands of the template nucleotide sequence. The primer extension sequences are then separated from the template sequences, and the process is repeated until the desired level of amplification is obtained. In subsequent cycles, the primer extension sequences serve as new templates for synthesizing the desired nucleotide sequence.

More specifically, the sample containing the template DNA nucleotide sequence is heated in the presence of the four different nucleoside triphosphates and the primer pair for an effective time and at an effective temperature to denature the DNA in the sample. Each oligonucleotide primer is sufficiently complementary to different strands of the template nucleotide sequence to hybridize with them, such that an extension sequence is synthesized from each oligonucleotide primer. When separated from its complement, the extension sequence serves as a template for the synthesis of the extension sequence of the other oligonucleotide primer. Preferably, the nucleoside triphosphates are deoxyribonucleoside triphosphates.

The denatured DNA is then cooled to a temperature that promotes hybridization, i.e., annealing, of each oligonucleotide primer to its complementary strand.

The natured DNA is contacted with a thermostable enzyme that catalyzes the combination of the nucleoside triphosphates to form primer extension products complementary to each strand of DNA. The thermostable enzyme is preferably a polymerase, such as Taq polymerase. The thermostable enzyme can be added after the denaturing or annealing steps or at the same time.

This mixture is maintained at an effective temperature and for an effective time to promote the activity of the enzyme and to synthesize an extension sequence of each oligonucleotide primer that is complementary to each strand of the template DNA sequence. The temperature must not be so high as to separate each extension product from its complementary strand at this point. The temperature can range from 55° C.-85° C. and the is 0.5 minutes to 4 minutes.

These steps are then repeated with the primer extension sequences that result in the amplification in the quantity of the template nucleotide sequence. This step of primer extension and the prior step of annealing may be carried out simultaneously or sequentially. The cycle of denaturing, annealing, and primer extension is carried out at as many time as required to prepare a sufficient number of double stranded DNA sequences.

As used herein, the term “primer” refers to an oligonucleotide, whether naturally occurring or synthetically produced, which is capable of acting as a point of initiation of synthesis when placed under conditions in which synthesis of a primer extension sequence complementary to the template sequence is induced. Such conditions include the presence of nucleotides (such as the four standard deoxyribonucleotide triphosphates) and an agent for polymerization such as a DNA polymerase, and suitable temperature and pH. Generally, each primer used in this invention will have from 10 to 40 nucleotides, and preferably, it has from 15 to 25 nucleotides.

All of this is preferably done in a small container, such as a cuvette. A cuvette provides a practical approach to allowing PCR technology to be practiced routinely by technicians and those of lesser skills, in an accurate fashion.

Any source of nucleic acid, in purified or nonpurified form, can be utilized as the template nucleotide sequence. Thus, the process may employ, for example, DNA or RNA, including messenger RNA, which DNA or RNA may be single stranded or double stranded. The template nucleotide sequence to be amplified may be only a fraction of a larger molecule or can be present initially as a discrete molecule, so that the specific sequence constitutes the entire nucleic acid. The starting nucleic acid may contain more than one desired specific nucleic acid sequence which may be the same or different. For example, in the present invention the template nucleotide sequence must include at least one recognition sequence for a restriction nuclease, wherein the restriction nuclease cuts in an offset fashion to produce ends having an overhanging piece of a single-stranded nucleotide sequence.

The template nucleotide sequence may be obtained from any source, for example, from plasmids, from cloned DNA or RNA, or from natural DNA or RNA from any source, including bacteria, yeast, viruses, and higher organisms such as plants or animals.

Any template nucleotide sequence can be amplified, as long as a sufficient number of bases at both ends of the sequence be known in sufficient detail so that two oligonucleotide primers can be prepared which will hybridize to different strands of the desired sequence and at relative positions along the sequence such that an extension sequence can be synthesized from one primer, when it is separated from its template (complement), can serve as a template for extension of the other primer into a nucleic acid of defined length. The greater the knowledge about the bases at both ends of the sequence, the greater can be the specificity of the primers for the template nucleotide sequence. The oligonucleotide primers may be prepared using any suitable method, such as, for example, the phosphotriester and phosphodiester methods described above, or automated embodiments thereof. In one such automated embodiment diethylphosphoramidites are used as starting materials and may be synthesized as described by Beaucage et al., Tetrahedron Letters (1981), 22:1859-1862. One method for synthesizing oligonucleotides on a modified solid support is described in U.S. Pat. No. 4,458,066, the contents of which are incorporated by reference herein.

If the template nucleotide sequence contains two strands, it is necessary to separate the strands before it can be used as the template, either as a separate step or simultaneously with the synthesis of the primer extension sequence. This strand separation can be accomplished by any suitable denaturing method including physical, chemical or enzymatic means. One physical method of separating the strands of the nucleic acid involves heating the nucleic acid until it is completely (>99%) denatured. Typical heat denaturation may involve temperature ranging from about 80° C. to 105° C. for times ranging from about 1 to 10 minutes. If the original template nucleotide sequence is single stranded, its complement is synthesized by adding one or two oligonucleotide primers thereto. If an appropriate single primer is added, a primer extension product is synthesized in the presence of the primer, an agent for polymerization and the four nucleotides described below.

When the complementary strands are separated, whether the template nucleotide sequence was originally double or single stranded, the strands are ready to be used as a template for the synthesis of additional nucleic acid strands. This synthesis can be performed using any suitable method. Generally it occurs in a buffered aqueous solution, preferably at a pH of 7-9, most preferably about 8. As a practical matter, the amount of primer added will generally be in molar excess over the amount of template/complementary strand.

The deoxyribonucleoside triphosphates dATP, dCTP, dGTP and TTP are also added to the synthesis mixture in adequate amounts and the resulting solution is heated to about 90-100° C. for from about 1 to 10 minutes, preferably from 1 to 4 minutes. After this heating period the solution is allowed to cool to from 20′-40° C., which is preferable for the primer hybridization. To the cooled mixture is added an agent for polymerization, and the reaction is allowed to occur under conditions known in the art. This synthesis reaction may occur at from room temperature up to a temperature above which the agent for polymerization no longer functions efficiently. Thus, for example, if DNA polymerase is used as the agent for polymerization, the temperature is generally no greater than about 45° C.

The agent for polymerization may be any compound or system, which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heatstable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There may be agents, however, which initiate synthesis at the 5′ end and proceed in the other direction, using the same process as described above.

As stated heretofore, preferably, primers or nucleotide bases used to replicate the template nucleotide sequence are labeled for later detection of helicase reaction products. For example, any appropriate signal-generating moiety may be used and the technology for attaching a signal generating moiety is known. For example in “Efficient Methods for Attachment of Thiol Specific Probes to the 3′ End of Synthetic Oligodeoxyribonucleotides,” Vol. 15 of Nucleic Acids Research, p. 5303 (1987), the techniques useful for the 3′ end attachment are discussed. The articles discussing 5′ end attachment are legion, for which the following is only representative: “Introduction of 5′ Terminal Functional Groups . . . . ,” Vol. 164 of Analytical Biochemistry, p. 336 (1987). It will be readily apparent that either the 3′ or the 5′ end can be used to attach the signal-generating moiety.

The newly synthesized double stranded nucleotide sequence is processed by a restriction nuclease to cut the double stranded nucleotide sequence at specific and recognizable sequence of nucleotides. For the present invention, the restriction nuclease has to cut in an offset fashion to provide the necessary overhanging pieces of the single-stranded DNA required for use in a helicase assay. There are numerous restriction nucleases available and one skilled in the art can determine the appropriate desired length of the bases in the overhang single-stranded sequence and select the appropriate restriction nuclease. A list of available restriction nucleases can be located at http://www.thelabrat.com/restriction/enzymesA.shtml, which provides information of the required recognizable sequences and the length of overhanging bases.

The double stranded nucleotide fragments having either a 5′ end or 3′ end, preferably has an overhang from about 1 to 10 bases, and more preferably from about 3 to 5 bases, are now available for use in a helicase assay to determine if an enzyme has helicase activity. The reaction conditions suitable for testing an enzyme suspected of having helicase activity and for separating the strands of nucleic acids with helicases are described by Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67).

### EXAMPLES

Materials and Methods

Oligonucleotide-Based Helicase Substrate Preparation

Oligonucleotide DF54 (5′-GGGACGCGTCGGCCTGGCACGTCGGCCGCTG CGGCCAGGCACCCGATGGCGTTT-3′ ) (SEQ ID NO: 1) was labeled using [γ-³²P]ATP and T4 polynucleotide kinase. Labeling reactions were stopped by adding EDTA to a final concentration of 25 mM. The labeled DF54 oligonucleotide was hybridized to DF50c oligonucleotide (5′-GCCATCGGGTGCCTGGCCGCAGCGGCCGACGTGC CAGGCCGACGCGTCCC-3′) (SEQ ID NO: 2) at 1:2 molar ratio in a buffer containing 40 mM Hepes-NaOH (pH=7.5) and 50 mM NaCl by heating to 100° C. for 3 mm followed by slow cooling to 25° C. Unincorporated [γ-³²P]ATP and unannealed oligonucleotides were removed using the following procedure. After hybridization, a 6× DNA loading buffer (0.1% xylene cyanol, 0.1% bromophenol blue and 50% glycerol) was added to a final concentration of 1×, and the mixture was fractionated through an 8% native polyacrylamide gel for 1 h at 100 V in 0.5× TBE (45 mM Tris, 4.5 mM boric acid and 0.5 mM EDTA). The substrate was located by autoradiography, the product was excised from the gel and sliced into small pieces and eluted in 3 vol of an elution buffer (0.5 M ammonium acetate, 10 mM magnesium acetate, 1 mM EDTA, pH=8.0) by incubating at 37° C. for 2 h. After centrifugation, the supernatant was collected, and the insoluble material was extracted once more with elution buffer. Following centrifugation, both supernatant fractions were combined, and the DNA was ethanol precipitated and dissolved in TE (10 mM Tris-HCl, pH=7.5, 1 mM EDTA). Specific activity of substrate was determined by liquid scintillation counting.

PCR-Based Helicase Substrate Preparation

Two PCR primers flanking a part of the pBluescript SK⁺ multiple cloning site were used to generate the DNA fragments used for the preparation of the helicase substrate. Using the primers SAC (5′-GAGCTCCACCGCGGTGGC-3′, map position 743-760) (SEQ ID NO: 3) and primer KPN (5′-GGTACCGGGCCCCCCCTC-3′, map position 653-670) (SEQ ID NO: 4) resulted in a PCR fragment of 108 bp. Prior to the PCR reaction, 10 pmol of the SAC primer was ³²P-labeled in a 10 μl reaction mixture containing 1× T4 polynucleotide kinase buffer, 16.6 pmol of [γ-³²P]ATP (3000 Ci/mmol, GE Biosciences) and 5 U of enzyme (Fermentas). The mixture was incubated at 37° C. for 30 min and directly added to the PCR reaction. The PCR reaction (50 μl) was performed with Pyrococcus furiosus (Pfu) polymerase (Stratagene) in 1× Pfu buffer in the presence of 5 ng pBluescript SK⁺ as template and 10 pmol of the labeled SAC primer and 10 pmol of KPN primer. For experiments using labeled nucleotides in lieu of the labeled primer, 50 pmol of [α-³²P]dCTP (6000 Ci/mmol, GE Biosciences) was added to PCR reaction containing 0.05 mM dNTPs. For both PCR reactions, the products were purified by a QIAprep PCR purification kit (Qiagen) in order to remove excess primer and nucleotides. Specific activity of substrate was determined by liquid scintillation counting.

DNA Helicase Assay

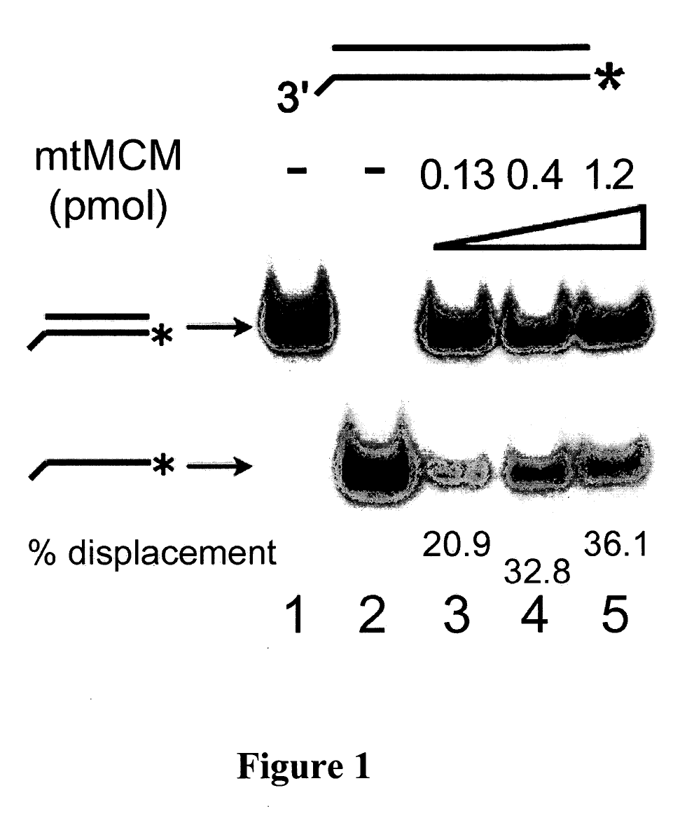

Methanothermobacter thermautotrophicus minichromosome maintenance (MCM) helicase activity was measured as described previously (8,9) in reaction mixtures (15 μl) containing 20 mM Tris-HCl (pH=8.5), 10 mM MgCl₂, 2 mM DTT, 100 μg/ml BSA, 5 mM ATP, 10 pmol of substrate and proteins as indicated in FIG. 1. Two oligonucleotides were used to generate a helicase substrate with a 4-base ssDNA overhang. Helicase assays were performed as described in Material and Methods in 15 μl reactions with the indicated concentrations of protein and 10 pmol substrate. The 32P-labeled oligonucleotide is marked with an asterisk. After incubation at 60° C. for 30 min, reactions were stopped by adding 5 μl of 5× loading buffer (100 mM EDTA, 1% SDS, 0.1% xylene cyanol, 0.1% bromophenol blue and 50% glycerol), and aliquots were fractionated on an 8% native polyacrylamide gel in 0.5× TBE and electrophoresed for 1.5 h at 150 V at 4° C. The helicase activity was visualized and quantitated by phosphorimaging. Lane 1, substrate only; lane 2, boiled substrate; lanes 3-5 contain 0.13, 0.40 and 1.2 pmol of proteins (as monomer), respectively. As can be seen as the helicase activity was very effective is unwinding

Helicase activities of the mesophilic enzymes (SV40 Large T-antigen, PriA, Rep, RecG, UvrD, RecQ) were measured in reaction mixtures (15 μl) containing 20 mM Tris-HCl (pH=7.5), 10 mM MgCl2, 2 mM DTT, 100 μg/ml BSA, 5 mM ATP, 10 fmol of substrate and 1.2 pmol of enzyme. Mixtures were incubated at 37° C. for 30 min and analyzed as described for the M. thermautotrophicus MCM.

A number of DNA helicases were shown to require only a short ssDNA overhang to initiate DNA unwinding. Examples are given in Table 1, below and in FIG. 1, which show that the M. thermautotrophicus MCM helicase required a minimum of 4 bases of 3′ overhang for helicase activity. Therefore, one may suggest that a helicase substrate could be generated using restriction digest of DNA molecules to generate a 4 nt 3′ or 5′ ss DNA overhang. Thus, PCR can be used to generate a labeled product that upon digestion can serve as helicase substrate. This would make substrate preparation simpler in comparison with other techniques and ease preparation of long DNA substrates.

TABLE 1

| Helicase | Polarity |
|---|---|
| Calf DNA helicase E | 3′→5′ |
| E. coli DNA helicase II | 3′→5′ |
| E. coli RecB | 3′→5′ |
| E. coli RecQ | 5′→3′ |
| E. coli Rep helicase | 3′→5′ |
| M. thermautotrophicus MCM | 3′→5′ |
| SV-40 large T-antigen | 3′→5′ |

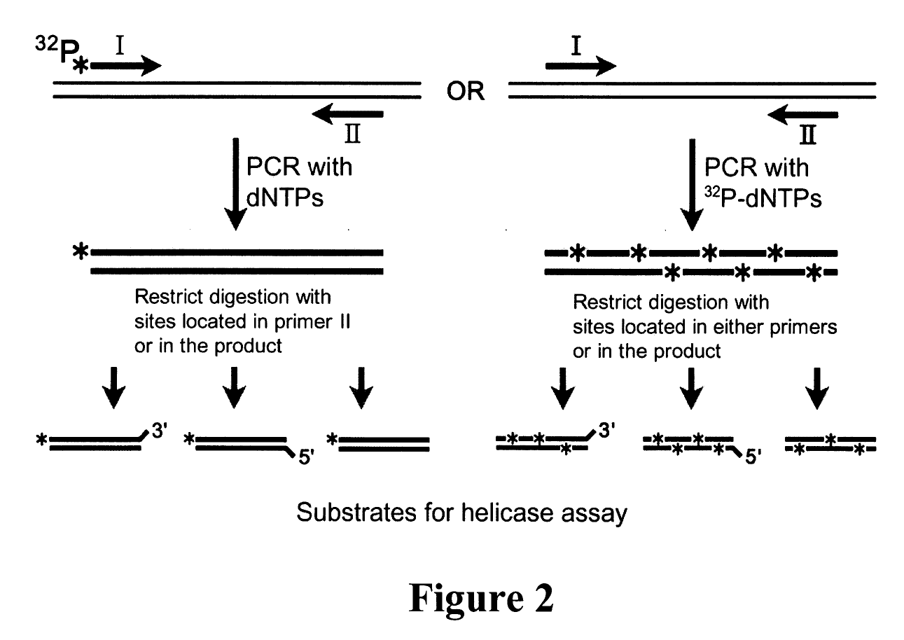

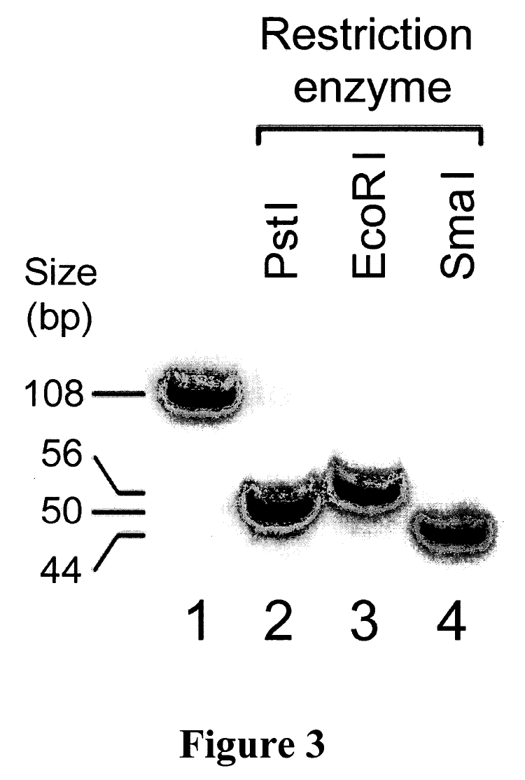

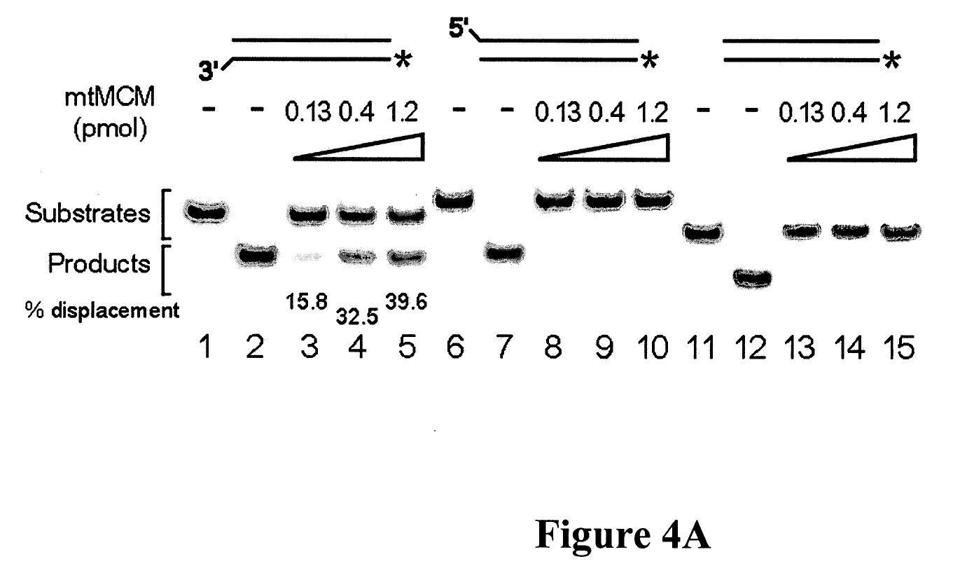

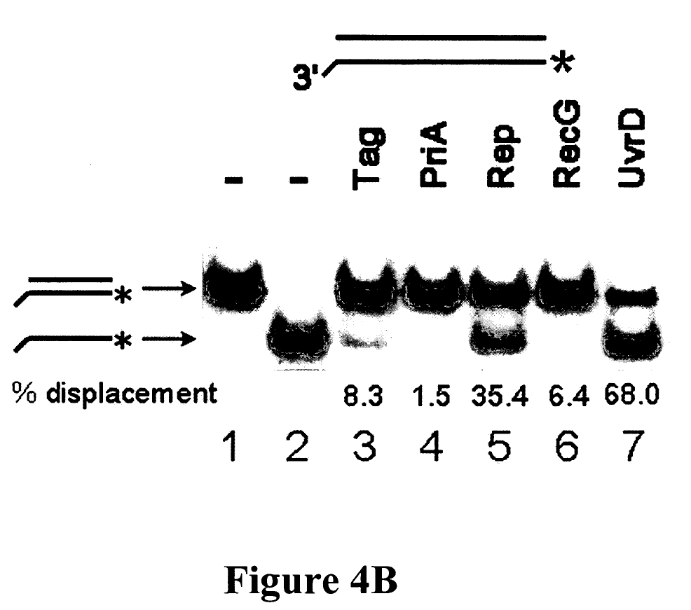

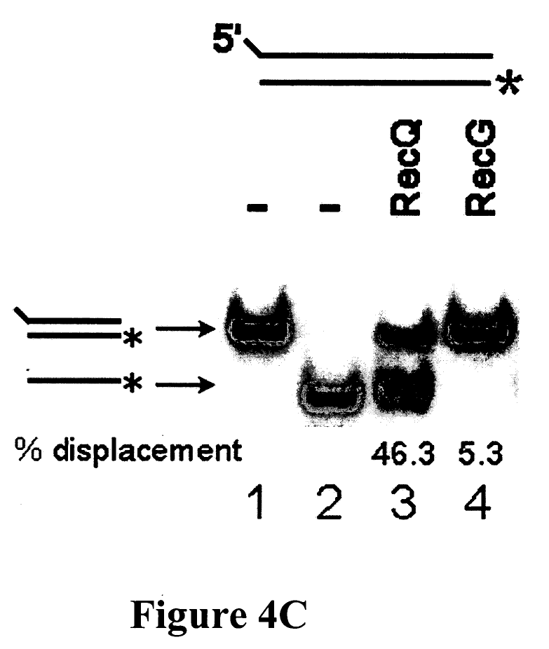

To determine whether a restriction enzyme-digested PCR product can serve as a helicase substrate, a protocol, summarized in FIG. 2 was developed. A PCR reaction was performed with either ³²P-labeled primer (only one primer labeled) or in the presence of ³²P-dNTPs (see Materials and Methods). Pfu polymerase was used for the reaction, as this enzyme produces a blunt-ended product. On the other hand, Thermus aquaticus (Taq) DNA polymerase resulted in the addition of an adenine residue at the 3′ end of the DNA and thus may serve as a substrate for helicases requiring only a single base 3′ overhang. Following PCR, the product was purified using QIAquick PCR purification kit (Qiagen) and digested with restriction enzymes that generate either a 3′ or a 5′ 4-base ssDNA overhang. The restriction sites can be located in the middle of the fragment or in the primers. Following digestion, the DNA can be used directly, without any further purification, in a helicase assay. The protocol was tested using a number of different restriction enzymes, locations (in the primers versus within the PCR products) and different PCR product sizes. One set of these products is shown here. A PCR product and its restriction fragment derivatives are shown in FIG. 3. PCR reactions were performed as described in Materials and Methods with ³²P-labeled primer, and products were purified using the QIAquick PCR purification kit (Qiagen) (lane 1) and digest with PstI (lane 2), EcoRI (lane 3) and SmaI (lane 4) restriction enzymes. PCR-based substrates were generated using the polylinker (SEQ ID NO: 5) of pBluescript SK⁺ (Stratagene) as template and two primers, one of which was ³²P-labeled, encompassing a part of the polylinker resulting in a 108 bp fragment as shown in FIG. 3, lane 1. The product was digested with either PstI restriction enzyme, resulting in a 50 bp fragment containing 4 bases of 3′ overhang (lane 2); EcoRI, yielding a 56 bp fragment containing 4 bases of 5′ overhang (lane 3) or SmaI restriction enzyme, resulting in a 44 bp blunt-ended fragment (lane 4). In each case, there is an additional fragment produced with similar overhang that is not labeled. These substrates were used in a helicase assay for the M. thermautotrophicus MCM helicase. As shown in FIG. 4A, the enzyme efficiently unwinds the substrate containing a 3′ overhang (lanes 3-5) but not the substrate containing a 5′ overhang (lanes 8-10) or the blunt-ended substrate (lanes 13-15). This is similar to the observations made with substrates containing longer ssDNA regions, either when oligonucleotides were annealed to ssM13 (10, 11) or to a longer oligonucleotide (12). The helicase was as efficient in unwinding the oligonucleotide-based substrate as the PCR-based substrate (cf. lanes 3-5 in FIGS. 1 and 4A). Other restriction enzymes have also been used with similar results (data not shown). These results demonstrate that a PCR-based helicase substrate is applicable for helicase studies.

M. thermautotrophicus MCM is active at high temperature (60° C.). Therefore, in order to determine whether the approach is applicable to helicases that are active at lower temperature and with different polarities, a number of additional helicases were analyzed. As shown in FIG. 4B, several helicases with 3′→5′ or 5′→3′ polarity (7) are active on the PCR-based substrate. However, as is evident from the results presented in FIG. 4C, this is by no means an approach suitable for all helicases. It is known that some helicases require a longer ssDNA region or even are active only on a fork-like DNA substrate.

### REFERENCES

The contents of all cited references are hereby incorporated by reference herein for all purposes
1. Delagoutte, E. and von Hippel, P. H. (2002) Helicase mechanisms and the coupling of helicases within macromolecular machines. Part I: Structures and properties of isolated helicases Q. Rev. Biophys., 35, 431-478.
2. Delagoutte, E. and von Hippel, P. H. (2003) Helicase mechanisms and the coupling of helicases within macromolecular machines. Part II: Integration of helicases into cellular processes Q. Rev. Biophys., 36, 1-69.
3. Venkatesan, M., Silver, L. L., Nossal, N. G. (1982) Bacteriophage T4 gene 41 protein, required for the synthesis of RNA primers, is also a DNA helicase J. Biol. Chem., 257, 12426-12434.
4. Matson, S. W., Tabor, S., Richardson, C. C. (1983) The gene 4 protein of bacteriophage T7. Characterization of helicase activity J. Biol. Chem., 258, 14017-14024.
5. Turchi, J. J., Murante, R. S., Bambara, R. A. (1992) DNA substrate specificity of DNA helicase E from calf thymus Nucleic Acids Res., 20, 6075-6080.
6. Matson, S. W. and Bean, D. W. (1995) Purification and biochemical characterization of enzymes with DNA helicase activity Methods Enzymol., 262, 389-405.
7. Tuteja, N. and Tuteja, R. (2004) Prokaryotic and eukaryotic DNA helicases. Essential molecular motor proteins for cellular machinery Eur. J. Biochem., 271, 1835-1848.
8. Shin, J. H., Grabowski, B., Kasiviswanathan, R., Bell, S. D., Kelman, Z. (2003) Regulation of minichromosome maintenance helicase activity by Cdc6 J. Biol. Chem., 278, 38059-38067.
9. Shin, J. H., Jiang, Y., Grabowski, B., Hurwitz, J., Kelman, Z. (2003) Substrate requirements for duplex DNA translocation by the eukaryal and archaeal minichromosome maintenance helicases J. Biol. Chem., 278, 49053-49062.
10. Chong, J. P., Hayashi, M. K., Simon, M. N., Xu, R. M., Stillman, B. (2000) A double-hexamer archaeal minichromosome maintenance protein is an ATP-dependent DNA helicase Proc. Natl Acad. Sci. USA, 97, 1530-1535.
11. Shechter, D. F., Ying, C. Y., Gautier, J. (2000) The intrinsic DNA helicase activity of Methanobacterium thermoautotrophicum ΔH minichromosome maintenance protein J. Biol. Chem., 275, 15049-15059.
12. Kelman, Z., Lee, J. K., Hurwitz, J. (1999) The single minichromosome maintenance protein of Methanobacterium thermoautotrophicum ΔH contains DNA helicase activity Proc. Natl Acad. Sci. USA, 96, 14783-14788.

That which is claimed is:

1. A method for generating a substrate for use in a helicase assay, the method comprising:
providing a PCR reaction composition comprising a template nucleotide sequence and at least one primer, wherein the primer includes a labeling tag;
hybridizing the primer to the template nucleotide sequence to form a template/primer complex;
separating the template/primer complex from unannealed template and primers;
digesting the template/primer complex with a restriction enzyme, wherein the restriction enzyme cuts in an offset fashion to produce ends having an overhanging piece of a single-stranded nucleotide sequence; ; and
separating double stranded fragments with a 3′ end or 5′ end overhang for use as a helicase substrate in the helicase assay.

2. The method according to claim 1, wherein the labeling tag is ³²P-labeled.

3. The method according to claim 1, wherein the 3′ end or 5′ end overhang comprises from about 1 to 10 bases.

4. The method according to claim 1, wherein the restriction enzyme is PstI or EcoRI.

5. The method according to claim 1, wherein the template nucleotide sequence comprises a sequence recognized by the restriction enzyme.

6. A substrate for use in a helicase assay, comprising:
a nucleotide template/primer double stranded complex comprising a recognizable nucleotide sequence by a restriction nuclease that upon restriction digestion of the nucleotide template/primer complex provides double stranded nucleotide sequence fragments having a 3′ end or 5′ end overhanging single stranded DNA sequences.

7. The substrate according to claim 6 wherein the 3′ end or 5′ end overhanging single stranded DNA sequences comprises from about 1 to 10 bases.

8. The substrate according to claim 6, wherein the substrate is used to test compounds for anti-helicase activity.

9. A method for generating a helicase substrate for use in a helicase activity assay, the method comprising:
a) providing a template nucleotide sequence having a recognition sequence for a restriction nuclease, wherein the restriction nuclease cuts in an offset fashion to produce ends having an overhanging piece of a single-stranded nucleotide sequence;
b) providing a first and second primer having a nucleotide sequence that will anneal to the 5′ and 3′ of the template nucleotide sequence, wherein the primers are sufficiently complementary to the template nucleotide sequence to hybridize therewith such that a first extension sequence synthesized from the first primer, when separated from its complement, can serve as a template for synthesis of a second extension sequence of the second primer;
c) amplifying the template nucleotide sequence by PCR amplification by combining at least the first and second primers, nucleotide bases and amplifying reagents to couple the nucleotide bases to the primers and generating the extension sequence complementary to the template nucleotide sequence to form a double stranded DNA sequence;
d) contacting the double stranded DNA sequence with the restriction nuclease to form double stranded DNA sequences having a 5′ or 3′ overhanging single stranded DNA end; and
e) using the double stranded DNA sequences having the 5′ or 3′ overhang end as a helicase substrate in a helicase assay.

10. The method according to claim 9 wherein the double stranded DNA sequences having either a 5′ end or 3′ end has an overhang from about 1 to 10 bases.

11. The method according to claim 10 wherein the double stranded DNA sequences having either a 5′ end or 3′ end has an overhang from about 3 to 5 bases.

12. The method according to claim 9, wherein at least one of the primers is labeled with signal tag, thereby providing detection of released helicase reaction product when the double-stranded DNA sequence is used in a helicase assay.

13. The method accord to claim 9, wherein the nucleotide bases incorporated into the extension sequences are labeled with a signal tag, thereby providing detection of released helicase reaction product when the double-stranded DNA sequence is used in a helicase assay.

14. The method according to claim 9, wherein the restriction nuclease is PstI or EcoRI.

15. A helicase assay kit for the detection of activity of suspected helicases, the kit comprising:
a template nucleotide sequence, a first and second primer, wherein the primers are substantially complementary to each strand of each specific nucleic acid sequence such that an extension sequence synthesized from one primer, when it is separated from its complement, can serve as a template for the synthesis of the extension product of the other primer;
an agent for polymerization;
four different nucleoside triphosphates; and
a restriction nuclease, where the wherein the restriction nuclease cuts in an offset fashion to produce ends having an overhanging piece of a single-stranded nucleotide sequence, thereby providing a helicase substrate having either a 5′ or 3′ overhanging single stranded DNA end and usable as a helicase substrate in the helicase assay to determine helicase activity of a suspected helicase whether it moves in either in the 3′→5′ or in the 5′→3′ direction.

16. The assay kit according to claim 15, wherein the restriction nuclease is PstI or EcoRI.

17. The assay kit according to claim 15, wherein at least one of the primers is labeled with signal tag, thereby providing detection of released helicase reaction product when the double-stranded DNA sequence is used in a helicase assay.

18. The assay kit according to claim 15, wherein at least one of the four different nucleoside triphosphates are labeled with a signal tag, thereby providing detection of released helicase reaction product when the double-stranded DNA sequence is used in a helicase assay.
